(19) United States
(12) Patent Application Publication
(10) Pub. No.: US 2020/0281737 A1
(43) Pub. Date: Sep. 10, 2020

(54) SPINAL IMPLANT AND METHOD FOR FORMING SPINAL IMPLANT

(71) Applicant: ADDIVATION MEDICAL, LLC, Red Bank, NJ (US)

(72) Inventors: Gregory Daniel STEBBINS, Hoboken, NJ (US); Gregory J. KOWALCYZK, Little Silver, NJ (US); Jose Luis Miranda CHARVET, Agoura Hills, CA (US); Luciano Bernardino BERTOLOTTI, Buenos Aires (AR); Christian Rutherford RUTT, Freehold, NJ (US); Marc S. MENKOWITZ, Rumson, NJ (US); Steven John PARAGIOUDAKIS, Ocean, NJ (US); Lawrence Matthew STANKOVITS, Rumson, NJ (US)

(73) Assignee: ADDIVATION MEDICAL, LLC, RED BANK, NJ (US)

(21) Appl. No.: 16/811,995

(22) Filed: Mar. 6, 2020

Related U.S. Application Data
(60) Provisional application No. 62/815,781, filed on Mar. 8, 2019.

(51) Int. Cl. A61F 2/44

(52) U.S. Cl. CPC A61F 2/442; A61F 2240/002

(57) ABSTRACT

In at least one embodiment there is an implant for a spine comprising a frame having at least one face having a plurality of ribs. Inside of the frame there is a lattice. In addition, there is at least one opening in the frame exposing said lattice, wherein said lattice extends beyond said frame to form greater interactions with adjacent bone structure. In addition there can also be a process for producing an implant comprising: determining a size of an implant frame; determining a size of an implant lattice; determining an orientation of the implant in a body; determining a first orientation of a lattice inside of the frame; graphically forming the implant; determining the opacity of the lattice inside of the frame from a first viewpoint; reorienting the lattice inside of the frame; determining the opacity of the lattice at a second orientation; determining which orientation results in lower opacity; and selecting the lattice orientation at a lower opacity.

### CROSS REFERENCE TO RELATED APPLICATIONS

This application is a non-provisional application of provisional application 62/815,781 filed on Mar. 8, 2019 the disclosure of which is hereby incorporated herein by reference in its entirety.

### BACKGROUND OF THE INVENTION

One embodiment of the invention relates to a spinal implant that is printed in a three-dimensional manner. The spinal implant can comprise an outer frame and a lattice. There is a need for a spinal implant that has a lattice orientation that allows for visual evaluation of the spinal implant once it is implanted into a user's body.

### SUMMARY OF THE INVENTION

In at least one embodiment there is an implant for a spine comprising a frame having at least one face having a plurality of supports such as ribs. Inside of the frame there is a lattice. In addition, there is at least one opening in the frame exposing said lattice, wherein said lattice extends beyond said frame to form greater interactions with adjacent bone structure.

In addition there can also be a process for producing an implant comprising: determining a size of an implant frame; determining a size of an implant lattice; determining an orientation of the implant in a body; determining a first orientation of a lattice inside of the frame; graphically forming the implant; determining the opacity of the lattice inside of the frame from a first viewpoint; reorienting the lattice inside of the frame; determining the opacity of the lattice at a second orientation; determining which orientation results in lower opacity; and selecting the lattice orientation at a lower opacity.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1A is a cephalad/caudad view of the frame of the spinal implant;

FIG. 1B is a anterior view of the spinal implant of FIG. 1A.

FIG. 2A is a side view which is either a-lateral view of the frame according to the orientation of FIG. 1A;

FIG. 2B is an oblique perspective view of the frame based upon the orientation of FIG. 1A;

FIG. 3A is a lateral view of the lattice portion of the implant;

FIG. 3B is a cephalad/caudad oblique view of the lattice portion of the implant based upon the orientation of FIG. 3A;

FIG. 4A is a cephalad/caudad view of the lattice portion of the implant based upon the orientation of FIG. 3B;

FIG. 4B is an anterior view of the lattice portion of the implant based upon the orientation of FIG. 4A;

FIG. 5 is a cephalad/caudad oblique view of the implant with both the frame and the lattice of the implant;

FIG. 6A is a cephalad/caudad view of frame and lattice portion of the implant based upon the orientation of FIG. 5;

FIG. 6B is an anterior view of the implant based upon the orientation of FIG. 6A;

FIG. 7 A is a lateral view of the implant with the section line A-A taken through it;

FIG. 7B is a cephalad/caudad cross-sectional view of the implant based upon the view of FIG. 7A;

FIG. 8 is a lateral view of the view shown in FIG. 7A;

FIG. 9A is an anterior view of a first embodiment of a first thickness;

FIG. 9B is a lateral view of the view of FIG. 9A;

FIG. 9C is an anterior view of a second embodiment of a second thickness;

FIG. 9D is a lateral view of the embodiment of FIG. 9C;

FIG. 9E is a anterior view of another embodiment;

FIG. 9F is a lateral view of the embodiment of FIG. 9E;

FIG. 9G is an anterior view of another embodiment;

FIG. 9H is a lateral view of the embodiment of FIG. 9G;

FIG. 9I is an anterior view of another embodiment;

FIG. 9J is a lateral view of the embodiment of FIG. 9I;

FIG. 10A is an anterior view of another embodiment;

FIG. 10B is a lateral view of the embodiment of FIG. 10A;

FIG. 10C is an anterior view of another embodiment;

FIG. 10D is lateral view of the embodiment of FIG. 10C;

FIG. 10E is a anterior view of another embodiment;

FIG. 10F is a lateral view of another embodiment;

FIG. 11A is a side view of a 3D image with the lattice at a first orientation;

FIG. 11B is another side view of the lattice at a second orientation rotated to provide greater visualization of bone growth;

FIG. 12A is a front perspective view of the lattice orientated in the frame;

FIG. 12B is a front perspective view of a second orientation of the lattice in the frame;

FIG. 13A is a side perspective view of a lattice structure;

FIG. 13B is a side view of a honeycomb cell of the lattice structure;

FIG. 14A is a view of a density profile of a first embodiment;

FIG. 14B is a view of a density profile of a second embodiment;

FIG. 15 is a flow chart of the process for rotating the lattice screen to provide an optimal image to show bone growth before printing the implant.

### DETAILED DESCRIPTION OF PREFERRED EMBODIMENTS

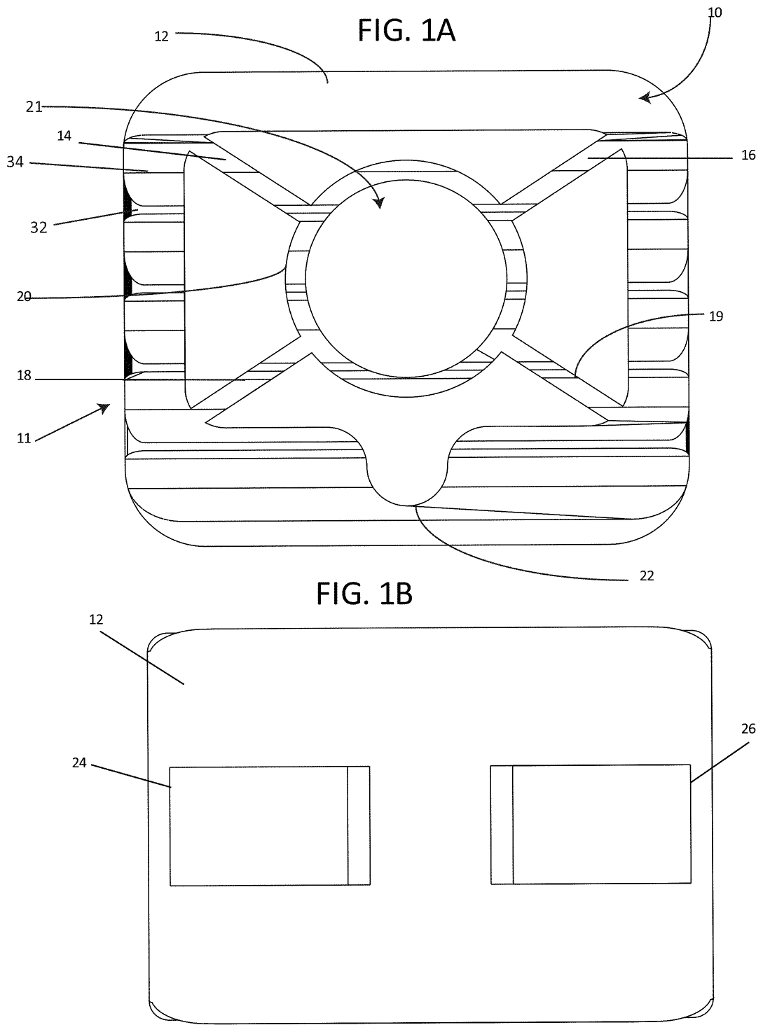

FIG. 1A is an anterior view of the frame of the spinal implant which forms a first embodiment 10. This embodiment includes a frame 11 which has a body section 12. Coupled to body section 12 are struts 14, 16, 18 and 19. In addition, these struts 14, 16, 18 and 19 are coupled to a central core 20 which forms a hub for the frame. Inside the core 20 is a hollow region 21. On a first or front face there are a plurality of supports such as ribs including ribs 32 with intermittent indents or valleys 34 between the ribs 32. In addition, there is an indent 22 in frame 11 which is configured to receive a lattice portion.

FIG. 1B is an anterior view of the spinal implant of FIG. 1A with this side being a view based upon the orientation of the frame in FIG. 1A. In this view there is frame 12 along with indents 24 and 26. Indents 24 and 26 are configured to allow for greater handling of the frame during implantation of the frame during surgery, or for handling of the frame with forceps prior to insertion. Each of these indents 24 and 26 are formed as ramp-shaped indents.

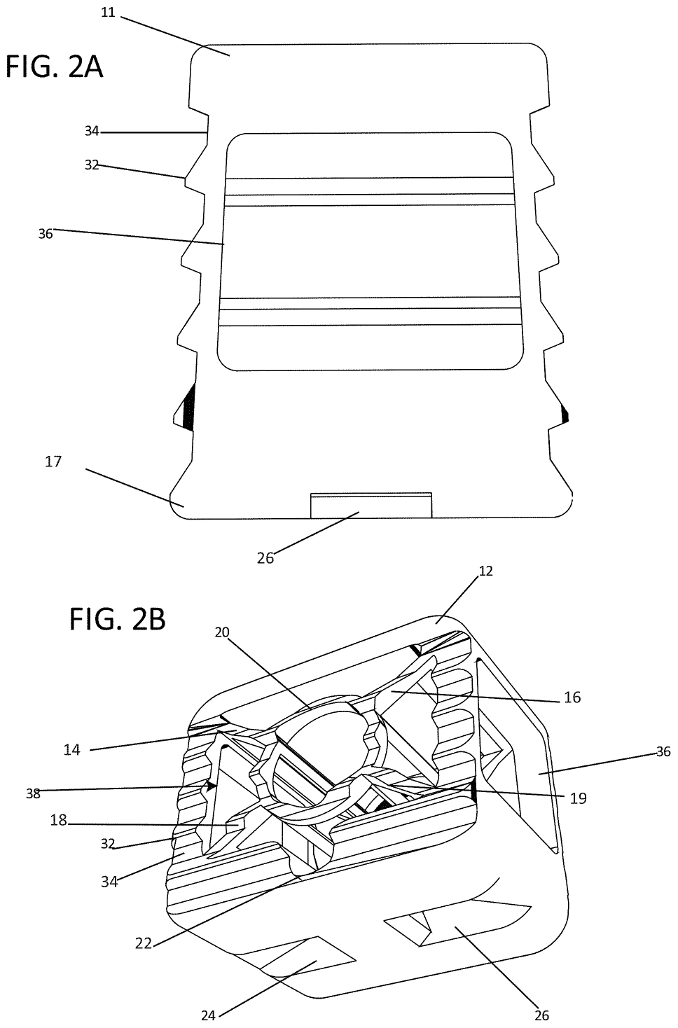

FIG. 2A is another lateral view which is either a left side view or a right-side view of the frame according to the orientation of FIG. 1A. With this view the frame 11 has ribs 32 as well as relative indents or valleys 34. There is also a side opening 36 which allows for side viewing inside of the frame. A bottom portion 17 is shown flared out forming an additional rib as well. While the terms “bottom”, “top”, “front”, “left”, and “right” are used, the implant can be implanted at any orientation and will most likely be implanted with the sides having ribs being positioned as top and bottom sides.

FIG. 2B is an oblique perspective view of the frame based upon the orientation of FIG. 1A. With this view there is shown frame portion 12 with struts 14, 16, 18 and 19 coupled to core 20. Ribs 32 are shown with flat front surfaces, indents or valleys 34. The bottom portion or face shows indents 24 and 26. Along the sides are windows or openings 36 and 38.

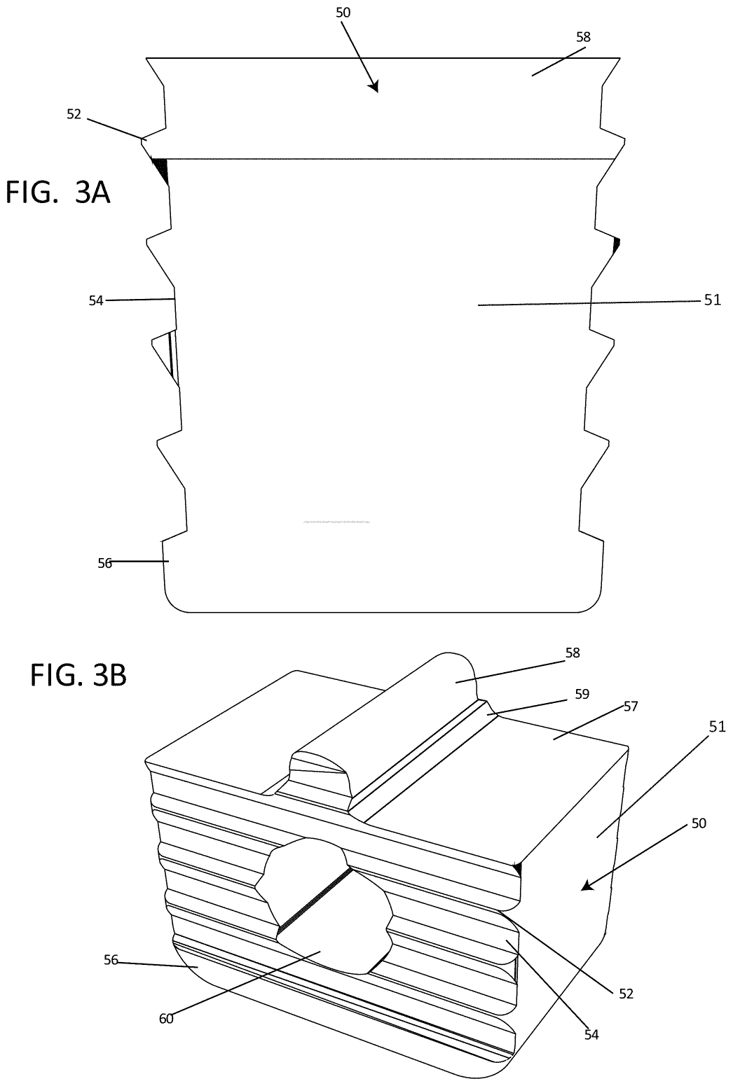

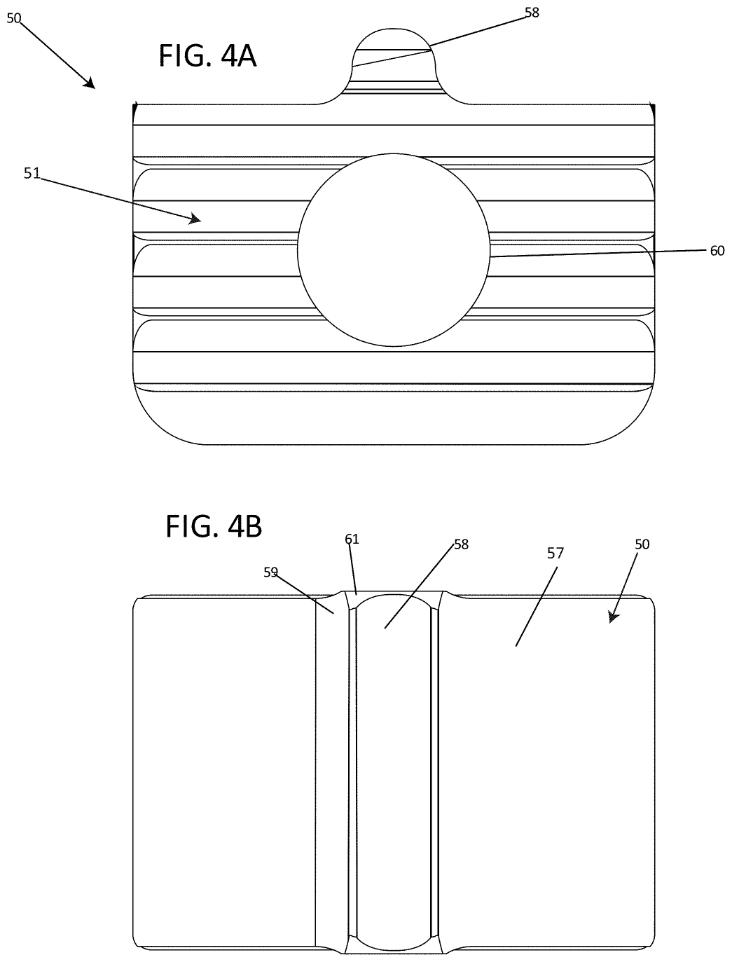

While the frame 11 with the outer frame portion 12 forms a first part of the implant there is another part of the implant which includes a lattice or screen. The lattice or screen is shown separate for purposes of display in FIGS. 3A-4B, however, when the device is printed both the frame and the lattice are printed together.

FIG. 3A is a lateral view of the lattice portion 50 of the implant 10. With this design, there is a body section 51 which includes at least one face having ribs 52 as well as a substantially flat surface 54 which sits between the ribs 52. There is also a base section 56 which forms a base block to the lattice. In addition, there is an extending portion 58 which extends up from the body section 51. This lattice portion can have cells that are formed as hexagonal shaped cells which when put together with other cells form a repeating 3-dimensional hexagonal shape.

FIG. 3B is an oblique view of the lattice portion 50 of the implant based upon the orientation of FIG. 3A. In this view body section 51 includes a face 57 which has extending arch portion 58 extending out from face 57 in an arch. In addition, there is an intermediate extending portion 59 which extends up from face 57 to extending portion 58. Ribs 52 are also shown, wherein these ribs extend between surface 54. There is also shown a central core opening 60 which is configured to receive or be coupled around core 20. The body section 51 can extend beyond a frame such as frame 12 so that it provides greater interaction between adjacent bones such as vertebrae.

FIG. 4A is a cephalad/caudad view of the lattice portion 50 of the implant based upon the orientation of FIG. 3B. This view shows body section 51, as well as central core 60. A top arch portion 58 is also shown.

FIG. 4B is an anterior view of the lattice portion of the implant based upon the orientation of FIG. 4A. In this view arch portion 58 is shown extending up from surface 57 along with extending portion 59. There is also an extending section 61 which extends out from a base of the arch.

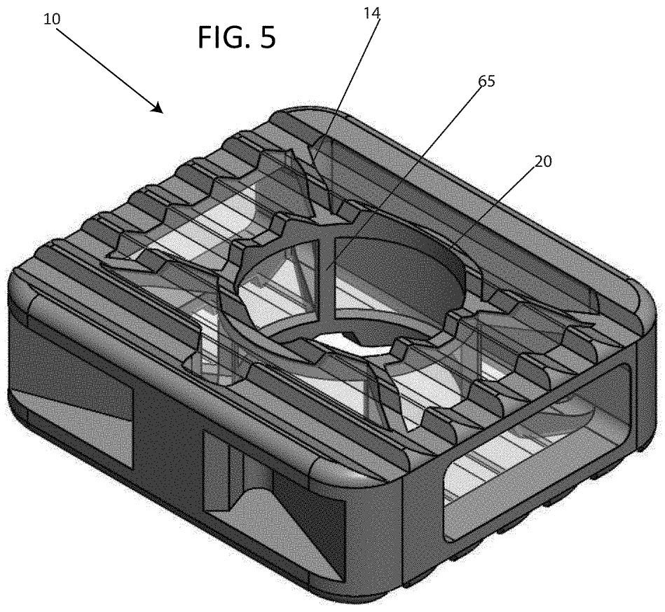

FIG. 5 is an oblique view of the implant 10 with both the frame and the lattice of the implant. The lighter portions of the implant 10 form the lattice section while the darker portions form the frame. With this view there is shown struts 14 as well as strut 65 which are each coupled to core 20. Strut 65 extends between the cores 20 on each face.

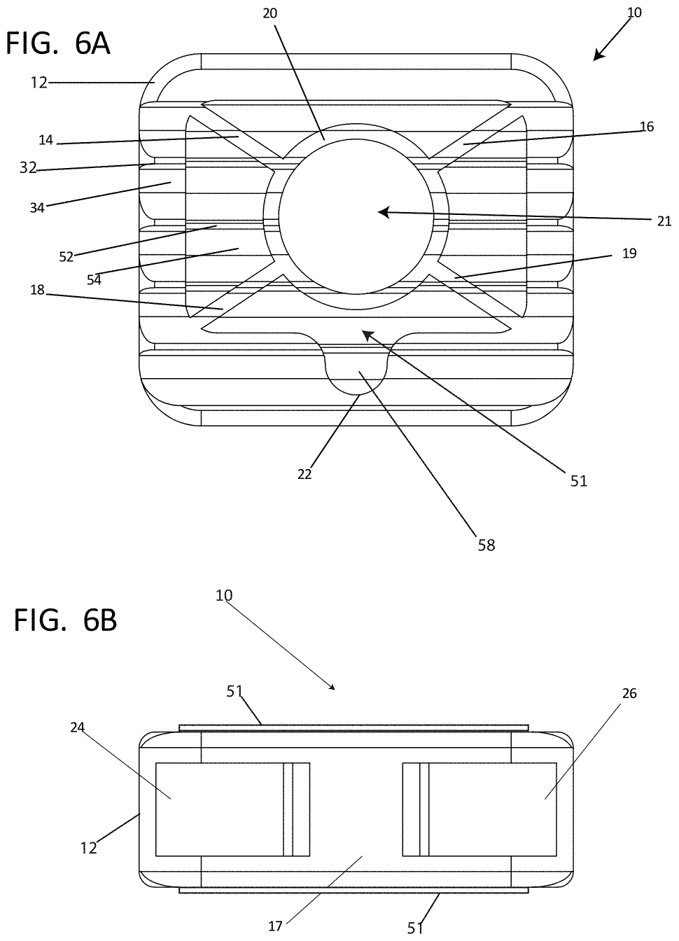

FIG. 6A is a cephalad/caudad view of frame and lattice portion of the implant 10 based upon the orientation of FIG. 1A. With this view there is shown frame portion 12 which includes struts 14, 16, 18 and 19 as well as core 20 along with lattice portion 51 interposed between the struts and core 20. In addition, extending section 22 extends into frame section 12 between indents 24 and 26 (See FIG. 6B) and in a region adjacent to non-indented portion 17. In addition, as shown in FIG. 6B the lattice structure 51 can be extended in a proud manner above the frame such as frame 12 to provide for greater bone on lattice structure. In addition, in at least one embodiment the porosity of the lattice structure can vary depending on the depth into the implant. This feature is shown in U.S. patent application Ser. Nos. 15/614,423, and 15/585,441, 15/665,097 and 16/268,074 the disclosure of these applications being hereby incorporated herein by reference in its entirety. In addition, in this view there is shown the lattice structure 51 extending beyond the extension of the frame such as frame 12.

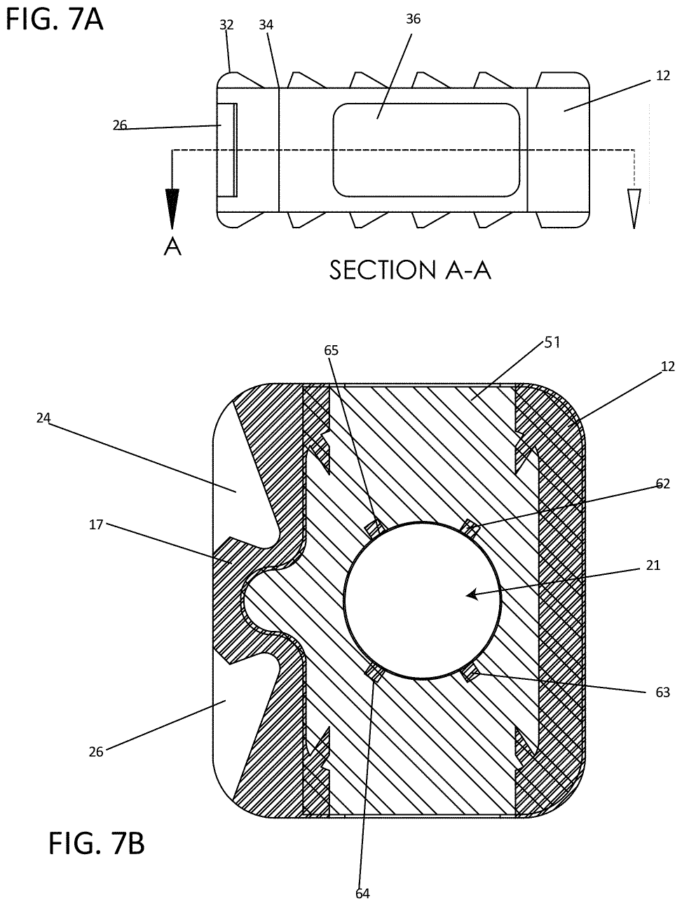

FIG. 7A is a lateral view of the implant with the section line A-A taken through it wherein this view includes ribs 32 as well as face portion 34. A side opening 36 is shown along with indent 26. A section line A-A is shown which is for the purpose of illustrating the cross-sectional view.

FIG. 7B is a side cross-sectional view of the implant based upon the view of FIG. 7 A wherein in this view there is a lattice 51 shown positioned inside of frame 12. A plurality of side struts 62, 63, 64, and 65 link the cores 20 together. Indents 24 and 26 are positioned on either side of non-indented portion 17. A hollow core section 21 is also shown.

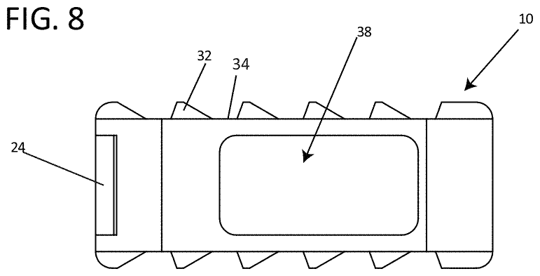

FIG. 8 is a lateral view of the view shown in FIG. 7 A which shows indent 24 as well as window opening 38. Ribs 32 are shown with flat face 34 positioned between these ribs.

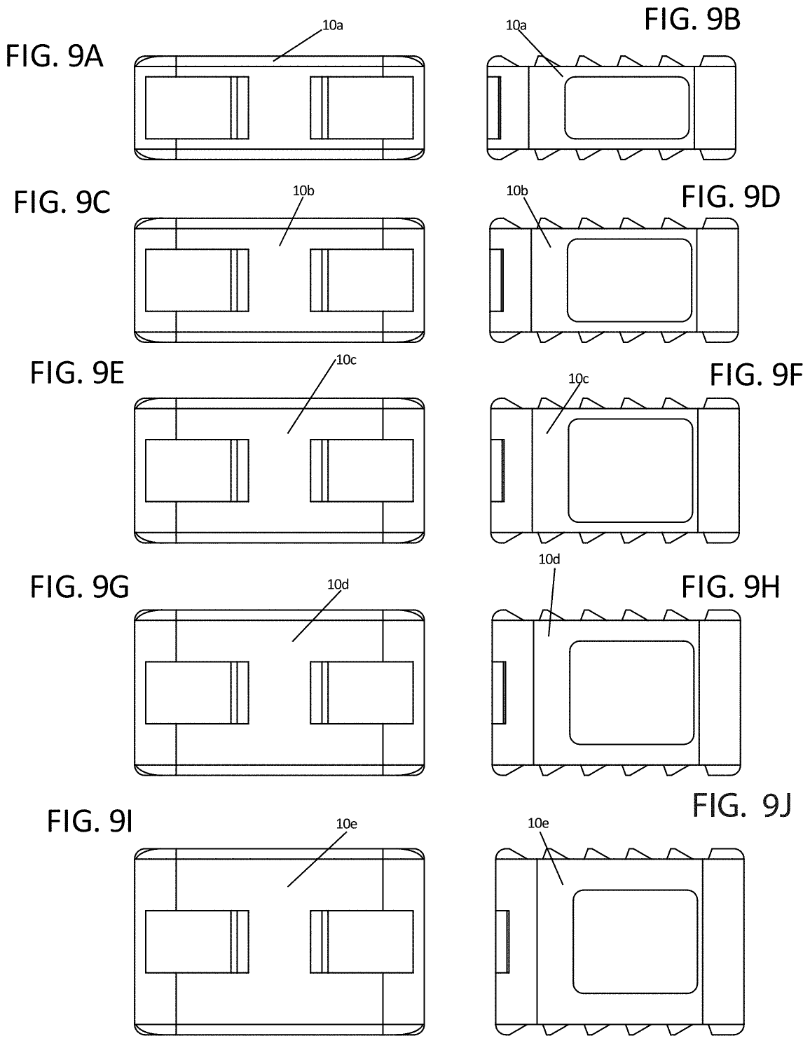

With these designs, there can be multiple different embodiments with different thicknesses. For example, there is a first embodiment with a first thickness shown in FIG. 9A as embodiment 10A. A lateral view of this embodiment is shown in FIG. 9B.

FIG. 9C is a view of a second embodiment of an implant 10b of a second thickness with FIG. 9D being an anterior view.

FIG. 9E is an anterior view of another embodiment 10c which shows another embodiment with FIG. 9F showing a lateral view of this embodiment 10c.

FIG. 9G is an anterior view of the embodiment 10d with FIG. 9H being a lateral view of this embodiment.

FIG. 9I is an anterior view of another embodiment 10e with FIG. 9J being a lateral view of this embodiment.

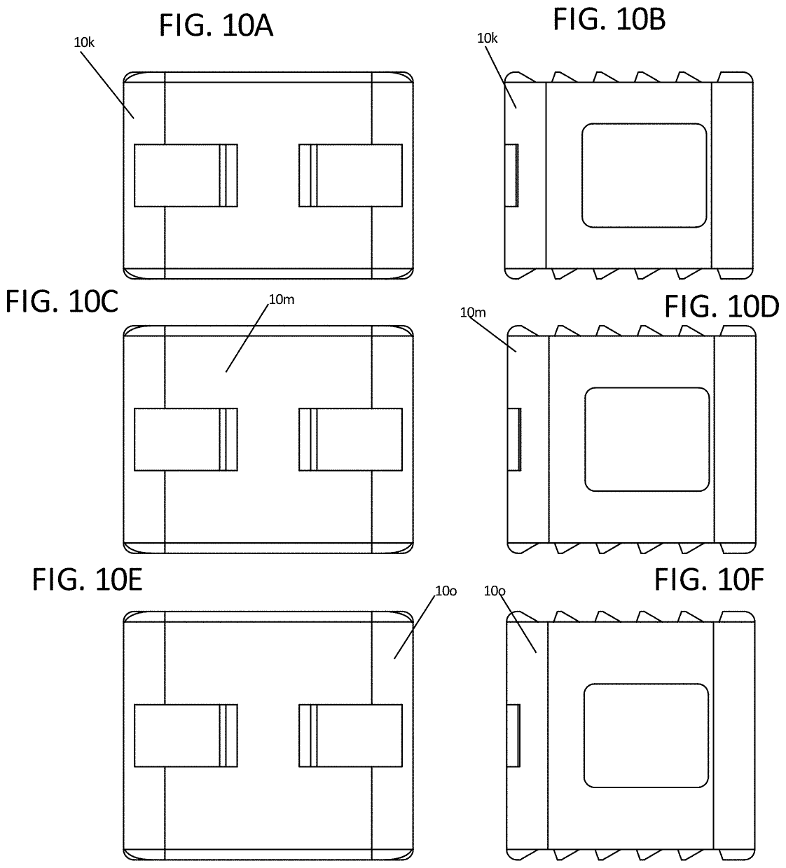

FIG. 10A is an anterior view of another embodiment 10k, with FIG. 10B being a lateral view of this embodiment.

FIG. 10C is an anterior side view of another embodiment 10m with FIG. 10D being a lateral view.

FIG. 10E is an anterior view of another embodiment 10o with FIG. 10F being a lateral view.

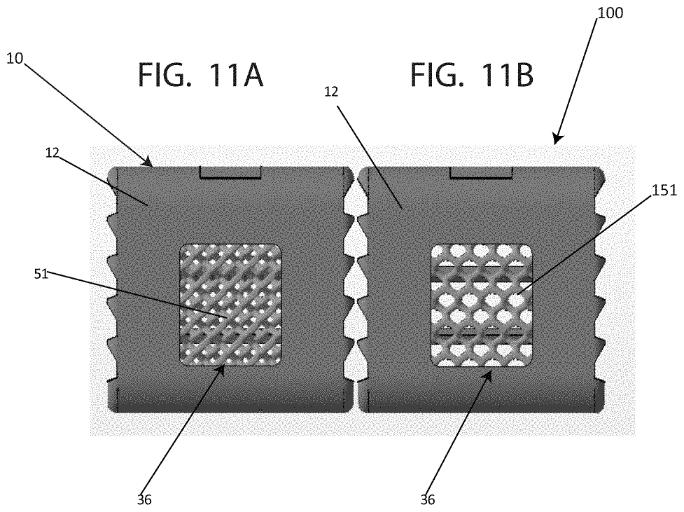

FIG. 11A is a lateral view of a 3D image with the lattice screen in a first orientation showing the lattice screen 51 disposed inside of the frame 12.

FIG. 11B is a lateral view of the 3D image with the lattice screen rotated to provide greater visualization of bone growth with this view lattice 151 is shown having much less opacity and more openings to view bone growth inside of the lattice screen.

Thus, after surgery, and after a period wherein there is a time for additional bone growth, a doctor can examine the extent of bone growth inside of the lattice via examination through either an X-ray, an MRI, Cat Scan or other visualization technique. Depending on the angle and orientation of the picture taken by one of these machines, the orientation of the lattice screen can be utilized to expose the extent of bone growth in the lattice.

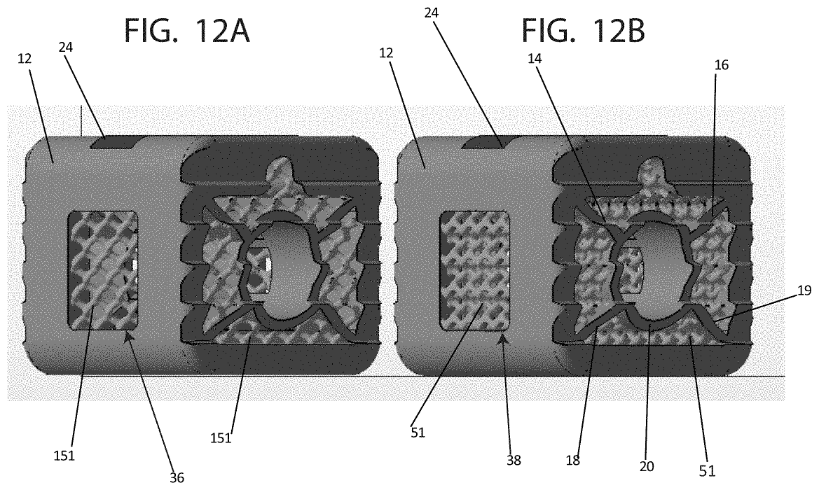

FIG. 12A is a front right perspective view of a 3D image with the lattice 151 orientated to show greater bone growth inside of frame 12.

FIG. 12B is a perspective view with the lattice 51 orientated in a view to show less bone growth through window or opening 38. This view shows struts 14, 16, 18 and 19 as well as core 20 as well. While a perspective view is shown, the most important view is from a side such as from the side views shown in FIGS. 11 and 12. In these lateral views a surgeon can determine optically through visual examination via either an X-ray, MRI scan or via a CAT scan the results of the bone growth in the lattice.

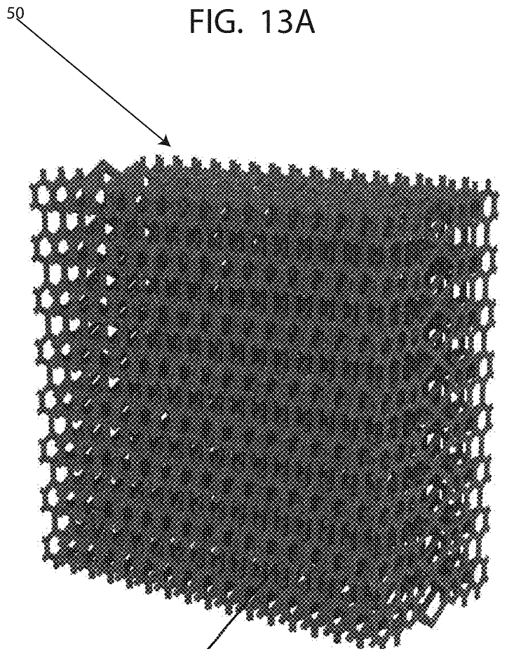

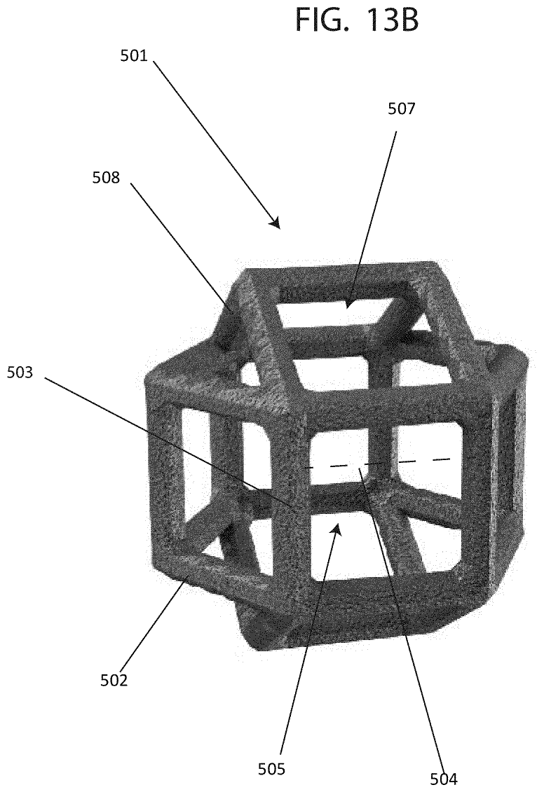

FIG. 13A is a side perspective view of a lattice structure 50 which includes a honeycomb 3-D pattern for different cells. A single two-dimensional cell can be in the form of a hexagonal shaped cell, however a three-dimension shell as shown in FIG. 13B as cell 501 includes a plurality of struts 502, 503, 508 which can form a face such as face 507. In addition, there is a core center region 505, as well as a distance across a face 504 which can be varied based upon the varying density of the lattice structure.

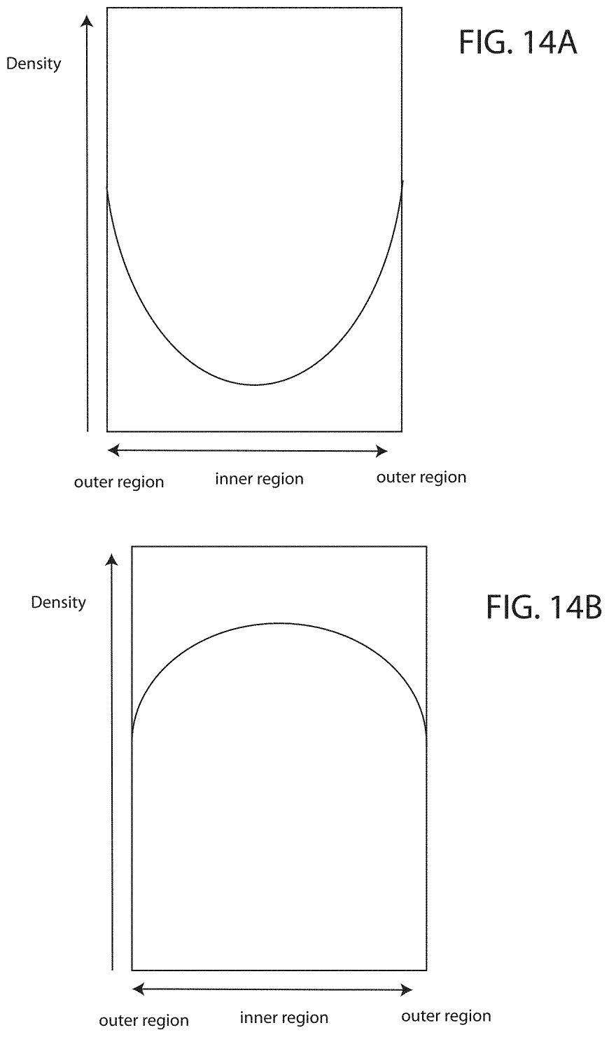

FIG. 14A is a view of a density profile of a first embodiment wherein the outer regions of the lattice such as lattice 50 are more dense than an inner region of the lattice structure which is interior to the external surfaces of the frame such as frame 12. The lower density of the central or inner regions of the lattice structure allow for greater insertion of bone growth material as well as providing greater room for bone growth. FIG. 14B shows an opposite profile with the density being lower at the exterior portions of the lattice such as towards the exterior portions of the frame.

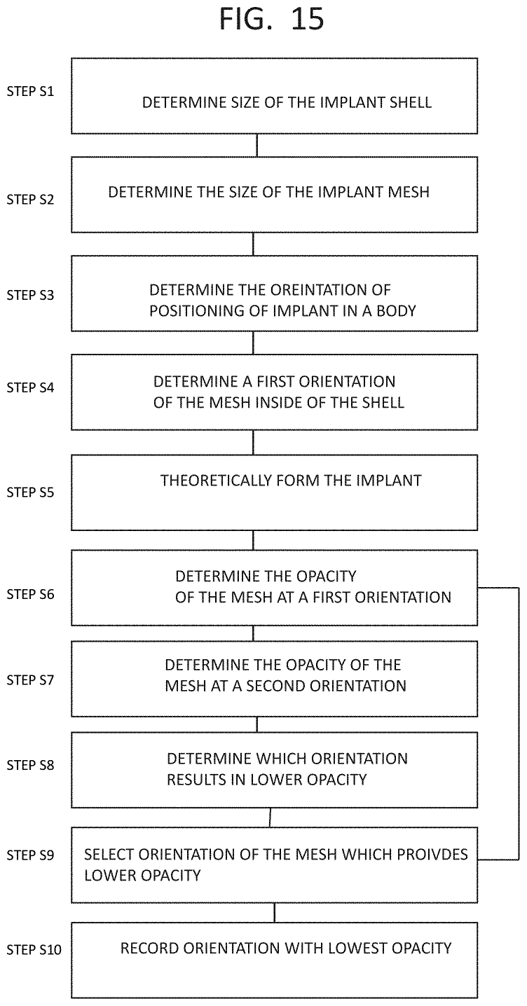

FIG. 15 is a flow chart of the process for rotating the lattice screen to provide an optimal image to show bone growth before printing the implant. For example, the process starts in step S1 wherein the user determines the size and shape of the implant frame. Next, the process involves determining the size of the implant lattice to fit inside of the implant frame. In at least one embodiment, the size of the implant lattice is determined to extend beyond the frame. The extension of the lattice such as lattice 51 or lattice 151 is at least partially beyond the frame to create greater interaction between bones and the lattice. The gaps in the lattice therefore allow for greater bone growth between the interstices in the lattice. In at least one embodiment, the extension of the lattice is at least 3 millimeters beyond the extension of the frame. In another embodiment the extension of the lattice is less than three millimeters such as two millimeters. In another embodiment the extension of the lattice is more than three millimeters such as four millimeters or five millimeters.

Next, in step S3 the user can determine the orientation and positioning of the implant in the body. For example, if the implant was to be positioned between two vertebrae in the body such as between C3 and C4 in the cervical spine, the frame would be if a first size. However, if the implant was to be positioned between two other vertebrae, such as between C4 and C5, then the thickness and size of the frame such as frame 12 would be different. These different sizes are shown in FIGS. 9A-10F.

Next, once the orientation of the implant is determined, the user using this system would then determine a first orientation of the lattice such as lattice 51 inside of the frame. Next, in step S5 the system could theoretically form the implant with the frame such as frame 12 being formed along with lattice such as lattice 51. Next, in step S6 the system could determine the opacity of the lattice at a first orientation. The system in step S7 would then model the design to determine the opacity of the lattice at a second orientation. Next, in step S8, the system would determine which orientation results in less or lower opacity. Next, in step S9 the system selects the orientation of the lattice which provides lower opacity. Steps S6-S9 can be repeated in a cycle until a desired level of opacity of the lattice is achieved. Next in step S10 the system records the orientation with lower opacity for printing.

Ultimately there is shown an implant for a spine such as a cervical, thoracic or lumbar spine section which can be implanted into the spine with an optimal level of opacity in the lattice to allow for greater visualization of the bone growth within a lattice.

Accordingly, while at least one embodiment of the present invention has been shown and described, it is to be understood that many changes and modifications may be made thereunto without departing from the spirit and scope of the invention as defined in the appended claims.

What is claimed is:

1. An implant comprising:
a frame having at least one face having a plurality of supports;
a lattice disposed inside of said frame; and
at least one opening in said frame exposing said lattice, wherein said lattice extends beyond said frame.

2. The implant as in claim 1, wherein the frame has at least one wall with at least one opening positioned in said at least one wall.

3. The implant as in claim 1, wherein the implant is for a spine and wherein the frame comprises a plurality of ribs and a plurality of struts and a core, with the core having a central opening.

4. The implant as in claim 2, wherein the frame comprises at least one additional wall with at least one indent in said at least one additional wall.

5. The implant as in claim 2, wherein the frame has at least one additional wall with at least one additional opening positioned in said at least one additional wall.

6. The implant as in claim 4, wherein the frame includes at least one interior indented portion positioned adjacent to said indented portion on said wall.

7. The implant as in claim 3, wherein the lattice comprises a body section and at least one central opening positioned around said core of said frame.

8. The implant as in claim 6, wherein the lattice includes an extending portion which extends into said interior indented portion of said frame.

9. The implant as in claim 1, wherein said lattice comprises ribs.

10. The implant as in claim 1, wherein said lattice is formed of cells having a hexagonal shape.

11. A process for producing an implant comprising:
determining a size of an implant frame;
determining a size of an implant lattice;
determining an orientation of the implant in a body;
determining a first orientation of a lattice inside of the frame;
graphically forming the implant;
determining the opacity of the lattice inside of the frame from a first viewpoint;
reorienting the lattice inside of the frame;
determining the opacity of the lattice at a second orientation;
determining which orientation results in lower opacity; and
selecting the lattice orientation at a lower opacity.

12. The process as in claim 11 further comprising the steps of:
forming said frame with a plurality of struts and a plurality of ribs.

13. The process as in claim 11, wherein the frame comprises at least one additional wall with at least one indent in said at least one additional wall.

14. The process as in claim 13, wherein the frame has at least one additional wall with at least one additional opening positioned in said at least one additional wall.

15. The process as in claim 12, wherein the frame includes at least one interior indented portion positioned adjacent to said indented portion on said wall.

16. The process as in claim 15, wherein the lattice comprises a body section and at least one central opening positioned around said core of said frame.

17. The process as in claim 16, wherein the lattice includes an extending portion which extends into said interior indented portion of said frame.

18. The process as in claim 17, wherein said lattice comprises ribs.

19. The process as in claim 18, wherein said lattice is formed of cells having a hexagonal shape.
